(19) United States
(12) Patent Application Publication
(10) Pub. No.: US 2024/0246266 A1
(43) Pub. Date: Jul. 25, 2024

(54) Method and Device for Manufacturing Microneedle Elements and a Microneedle Element

(71) Applicant: LTS Lohmann Therapie-Systeme AG, Andernach (DE)

(72) Inventors: Natasha Gannon, Koblenz (DE); Stefan Erlhofer, Ochtendung (DE)

(21) Appl. No.: 18/289,948

(22) PCT Filed: May 10, 2022
(86) PCT No.: PCT/EP2022/062657

§ 371 Date: Nov. 8, 2023

(30) Foreign Application Priority Data
May 10, 2021 (DE) 10 2021 112 136.1

(51) Int. Cl. B29C 39/02; A61M 37/00; B29C 39/40; B29L 31/00

(52) U.S. Cl. CPC B29C 39/025; A61M 37/0015; B29C 39/405; A61M 2037/0023; A61M 2037/0046; A61M 2037/0053; A61M 2207/10; B29L 2031/7544; B29L 2031/756; B29L 2031/759

(57) ABSTRACT

A method for manufacturing a microneedle element, including the following steps: providing a mold element for the microneedle elements to be manufactured, filling the mold element with a first formulation, curing the first formulation, and pressurizing the first formulation during curing. Furthermore, a microneedle element and a device for manufacturing a microneedle element.

### CROSS-REFERENCE TO RELATED APPLICATIONS

This application is the United States national phase of International Application No. PCT/EP2022/062657 filed May 10, 2022, and claims priority to German Patent Application No. 10 2021 112 136.1 filed May 10, 2021, the disclosures of which are hereby incorporated by reference in their entireties.

### BACKGROUND OF THE INVENTION

### Field of the Invention

The invention relates to a method and a device for manufacturing microneedle elements comprising at least one microneedle and to a microneedle element.

Microneedle elements, such as microarrays, have a plurality of microneedles that are typically arranged in a support element, such as a patch, a plaster or the like, or are connected to the same. On the other hand, a microneedle element may be a single microneedle or several individual microneedles. In general, microarrays have a high number of microneedles, the length of which is dimensioned such that, when being pushed into the skin of a patient, they penetrate into the skin only so far that the needle tips do not come into contact with nerves and vessels, if possible. Here, the needles comprise an active ingredient, for example a medication. The corresponding active ingredient may be provided on an upper side of the needles or may be provided in the needles. When the active ingredient is arranged in the needles, the needles or components of the needles are in general made of a material that dissolves in the skin of a patient. Dissolving microneedles may in particular have one or more formulations containing active ingredient and/or being free of active ingredient, preferably consist thereof.

### DESCRIPTION OF RELATED ART

Microneedles and/or microarrays are manufactured by using dies, for example, which have a plurality of mold openings formed as recesses. These mold openings serve as negative molds for the microneedles to be manufactured and/or the microarray to be manufactured.

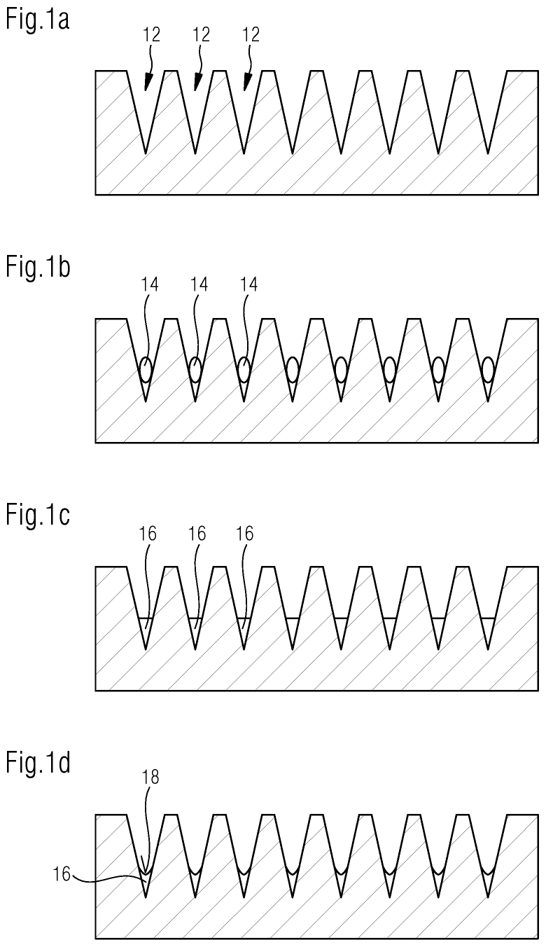

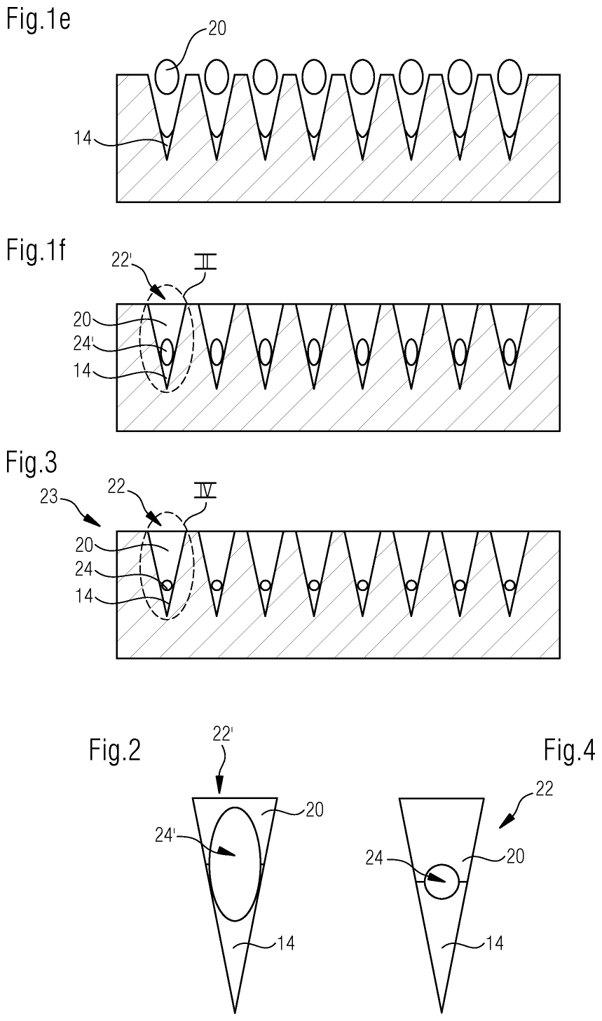

A conventional manufacturing method for microneedle elements is shown in FIGS. 1a-1f. FIG. 2 shows a detail view of FIG. 1f (II). The starting point is a die 10, e.g. a silicone die, as shown in FIG. 1a. The mold openings 12 of die 10 are first filled with a liquid formulation 14, in particular one that contains an active ingredient, which is also referred to as tip formulation (FIG. 1b). This results in the tips of the mold openings 12 (FIG. 1c) being filled with formulation 14. Tip formulation 14 is then cured by drying so that tips 16 of the microneedles to be manufactured are formed. Due to drying, there is a reduction in volume, in particular due to a reduction of a solvent, such as water and/or ethanol, of the formulation. As shown in FIG. 1d, the volume reduction can result in an uneven, e.g. concave, surface 18 of tip formulation 14. After tip formulation 14 has cured, the mold openings 12 are filled with a further formulation 20, in particular one that is free of active ingredients, also referred to as baking formulation (FIG. 1e). Backing formulation 20 is then cured, in particular by drying. Backing formulation 20 and tip formulation 14 are bonded. The bonded cured formulations 14, 20 together form a microneedle 22′ (FIG. 1f). The microneedles 22′ can be demolded, e.g. by means of an adhesive foil. When filling with baking formulation 20, air pockets 24′ often occur between baking formulation 20 and tip formulation 14. The illustrations in the Figures are schematic. For example, the dropshaped formulations 14, 20 in FIGS. 1b and 1e may not be depicted as large enough to be filled accordingly (according to FIGS. 1c and 1f). Also, the filling by means of individual drops is only to be seen schematically. For example, filling with several drops and/or a formulation jet is also possible. It is also possible, for example, to use a flow-in filling instead of or in addition to the drop-in filling shown.

The air pockets 24′ (see e.g. FIG. 2) may cause several problems. On the one hand, there may be no or insufficient bond between baking formulation 20 and tip formulation 14. On the other hand, there may be instabilities so that, for example, breaking occurs during demolding, handling or use.

### SUMMARY OF THE INVENTION

It is an object of the invention to provide a method and a device for manufacturing microneedle elements comprising at least one microneedle, with improved quality, in particular stability, of the microneedle elements to be manufactured. Another object of the present invention is to provide a microneedle element with improved quality, in particular stability.

According to the invention, the object is achieved with a method as described herein, a device as described herein, and a microneedle element as described herein.

The method according to the invention is a method for manufacturing microneedle elements, wherein a microneedle element to be manufactured comprises at least one microneedle. Preferably, the method according to the invention is in particular a method for manufacturing microarrays, wherein the microneedle element to be manufactured is a microarray having several microneedles connected in particular by means of a support element. On the other hand, it is also possible that the method according to the invention is a method for manufacturing microneedles, in particular individual microneedles. The first step of the method consists in providing a mold element for the microneedle elements to be manufactured. The mold element is in particular a die comprising silicone, for example. Preferably, the mold element comprises at least one mold opening formed as a negative mold for the microneedle element to be manufactured. A further step of the method consists in filling the mold element with a first formulation, in particular a backing formulation. After the mold element has been filled with the first formulation, the first formulation is cured. At least partially simultaneously with curing, the first formulation is pressurized during curing. It is preferred that filling with the first formulation is performed such that the mold opening is substantially completely filled with formulation. Due to the pressurization during curing, it is particularly advantageous that gas pockets, e.g. air pockets, can escape from the formulation and/or no gas pockets can be introduced. In this way, a more stable microneedle element can be manufactured. It is preferred that the mold openings are cylindrical or conical in shape. In particular, the base area of the cylindrical or conical mold opening is a circle, oval, rectangle or square. If the mold openings have a conical shape, they can also be truncated cones. The cross-section of the mold opening preferably tapers. In particular, the mold openings are symmetrical in the longitudinal direction, in particular rotationally symmetrical. It is preferred that the mold element is gas-tight or gas-permeable.

It is possible that only a single formulation is introduced, which thus forms in particular the entire microneedle element to be manufactured. However, it is preferred that the microneedle element to be manufactured is manufactured from several, in particular at least two formulations. Thus, it is preferred that before filling with the first formulation, the mold element is filled with a second formulation, in particular a tip formulation. Preferably, the following is thus implemented, in particular in the following order: i. providing the mold element; ii. filling the mold element with the second formulation; iii. filling the mold element with the first formulation; iv. curing with simultaneous pressurization of the first and preferably the second formulation. By curing the first formulation with simultaneous pressurization, it can thus be advantageously implemented that gas pockets, e.g. air pockets, can escape between the first and the second formulation. Additionally or alternatively, it is in particular advantageously implemented that gas pockets are compressed, preferably condensed, between the first and the second formulation. Compression in particular results in gas pockets, such as air bubbles, being smaller. After curing, expansion of the gas pockets is in general no longer possible. This has the advantage that there is a better bond, in particular a larger bonding surface, between the first and the second formulation. The method is preferably performed such that the pressure between the formulation, in particular the pressure of the at least one gas pocket, is preferably at least 2 bar, particularly preferred at least 3 bar.

In a preferred embodiment, after filling the mold element with the second formulation and in particular before filling the mold element with the first formulation, the second formulation is cured. It is possible that a pressurization takes place simultaneously with the curing of the second formulation.

It is possible that more than two formulations are used for manufacturing the microneedle element. Thus, in a preferred embodiment of the method, before the step of filling the mold element with the first formulation and after the step of filling the mold element with the second formulation, the further step is carried out at least once: Filling the mold element with at least one additional formulation, in particular an intermediate formulation, and preferably curing the at least one additional formulation, preferably with simultaneous pressurization.

It is preferred that the first and/or the second and/or the at least one additional formulation comprises a solvent, such as water, and/or liquid organic substances, and/or alcohol, preferably ethanol. In particular, the second formulation comprises at least one active ingredient. The first and/or the at least one additional formulation is preferably free of active ingredient.

In a preferred embodiment, when filling the mold element with the first formulation, the mold element is overfilled so that the first formulation emerges from the mold element. The overfill can then be removed or remain. If the overfill remains, it is possible to create a backing layer, for example.

It is preferred that the first and/or the second and/or the at least one additional formulation is/are cured by drying, in particular air drying. Preferably, the curing process is a drying process. It is possible that drying is realized by means of an air flow and/or infrared.

In a preferred embodiment, temperature is applied when curing the first and/or the second and/or the at least one additional formulation.

In a preferred embodiment, during the curing of the first and/or the second and/or the at least one additional formulation, a continuous pressurization of the first and/or the second and/or the at least one additional formulation takes place. In particular, the pressurization is carried out until the first and/or the second and/or the at least one additional formulation is at least partially, in particular completely, cured. Preferably, the pressurization takes place during at least a part of the duration of the curing of the first and/or the second and/or the at least one additional formulation. In particular, the pressurization takes place for at least one quarter, preferably half, particularly preferred three quarters of the duration of the curing of the first and/or the second and/or the at least one additional formulation. Preferably, the pressurization can take place during the entire duration of the curing of the first and/or the second and/or the at least one additional formulation. The pressurization and/or the curing of the first and/or the second and/or the at least one additional formulation is preferably carried out until the first and/or the second and/or the at least one additional formulation still has a maximum of 50%, preferably a maximum of 30%, particularly preferred a maximum of 10% of the original liquid content, in particular solvent content. Said liquid content in particular concerns the volume fraction and/or the mass fraction. It is preferred that the pressurization is carried out in particular until no further expansion of the gas pockets is possible due to the curing.

It is preferred that the pressurization, in particular of the first and/or the second and/or the at least one additional formulation, is carried out by means of overpressure. Overpressure here preferably means a gas pressure above ambient pressure. It is preferred that the overpressure is generated by means of an overpressure chamber, wherein the mold element is arranged at least partially, in particular completely, within the overpressure chamber. In particular, the overpressure is at least 1 bar, preferably at least 3 bar, and particularly preferred at least 3 bat above the ambient pressure. In a preferred embodiment, the overpressure does not exceed 10 bar, in particular does not exceed 5 bar. As an alternative or in addition to the pressurization by means of overpressure, the pressurization can be carried out mechanically, in particular by means of a pressing device comprising a punch and/or a roller.

In a preferred embodiment, the pressurization and/or the curing, in particular of the first and/or the second and/or the at least one additional formulation, takes place over a period of at least 30 minutes, preferably at least 60 minutes, particularly preferred at least 90 minutes.

In particular, the mold element comprises several microneedle negative molds. The several microneedle negative molds in particular form a microarray negative mold.

It is preferred that the mold element is filled with the first formulation such that the first formulation covers the several microneedle negative molds. In this way, a support element, also called backing, can advantageously be formed integrally with the first formulation.

The microneedle element according to the invention is in particular a microneedle or a microarray comprising at least one microneedle. The microneedle element is in particular obtainable by the method according to the invention described above. As an alternative or in addition to the definition that the microneedle element is obtainable by the method described above, the microneedle element according to the invention comprises at least two, preferably at least partially cured formulations. The formulations are bonded with each other. In particular, it is at least a tip formulation and a backing formulation. Preferably, at least one gas pocket, e.g. an air bubble, is arranged between the at least two formulations. Here, the gas pocket is above ambient pressure. Preferably, there is an overpressure between the formulations. The pressure between the formulation, in particular the pressure of the at least one gas pocket, is preferably at least 2 bar, particularly preferred at least 3 bar. In a preferred embodiment, the microneedle element comprises a cavity, preferably substantially spherical or ellipsoidal, between the first and second formulations. The cross-sectional area of the cavity is preferably smaller than the bonding surface between the first and the second formulation, in particular by at least 30%. The diameter of the cavity is in particular smaller than the diameter of the microneedle element at the junction between the first and the second formulation, in particular by at least 30%. In particular, it is advantageous that the cavity of the microneedle element according to the invention is smaller than that of previous microneedle elements. Furthermore, it is in particular advantageously implemented that the microneedle element according to the invention has a substantially spherical cavity, preferably with a circular cross-section, wherein previous microneedle elements have an ellipsoidal cavity.

The device according to the invention is a device for manufacturing a microneedle element comprising at least one microneedle. The device comprises a mold element for the microneedle element to be manufactured. The mold element is in particular configured as the above-described mold element for the method according to the invention. Furthermore, the device comprises a pressurization device. The pressurization device preferably comprises an overpressure chamber and/or a pressing device. Preferably, the pressurization device comprises a pump for generating overpressure, in particular in the overpressure chamber. It is preferred that the mold element is arranged at least partially, in particular completely, within the overpressure chamber. In a preferred embodiment, the device comprises a drying device, in particular generating heat and/or air flow. The drying device can be connected to the pressurization device, in particular integrally. The pressurization device is preferably configured and/or arranged such that a pressure is applied to the mold element. In particular, the pressure is applied at least to the mold opening of the mold element. The drying device can comprise a pump and/or a heating element, preferably generating infrared. It is preferred that the device comprises a filling device, preferably comprising a dosing device, for filling the mold element with at least one formulation.

In particular, the device is configured to perform the method according to the invention described above. Preferably, the device comprises one or more of the features described above in the context of the method, in particular the device features.

### BRIEF DESCRIPTION OF THE DRAWINGS

The terms FIG., FIGS., Figure, and Figures are used interchangeably in the specification to refer to the corresponding figures in the drawings.

In the following, the invention is described in more detail by means of a preferred embodiment with reference to the accompanying drawings.

### In the Figures:

FIGS. 1a-1f are schematically sectioned side views of a die in different conditions for illustrating a method for manufacturing microneedle elements according to prior art,

FIG. 2 is a detailed view according to II of FIG. 1f for illustrating a microneedle element in the form of a microneedle of prior art,

FIG. 3 is a schematically sectioned side view of a die in a condition for illustrating an embodiment of a method for manufacturing a microneedle element according to the invention,

FIG. 4 is a detailed view according to IV of FIG. 3 for illustrating an embodiment of a microneedle element according to the invention in the form of a microneedle,

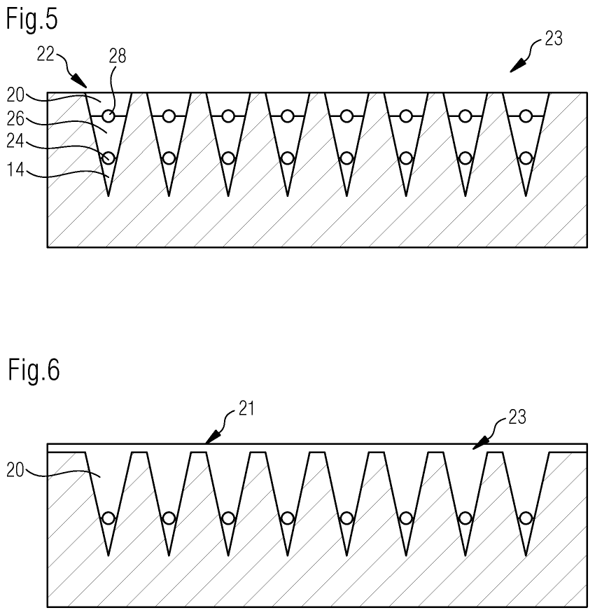

FIGS. 5-6 are schematically sectioned side views of a die in a condition for illustrating a further embodiment of a method for manufacturing microneedle elements according to the invention with further embodiments of microneedle elements according to the invention, and

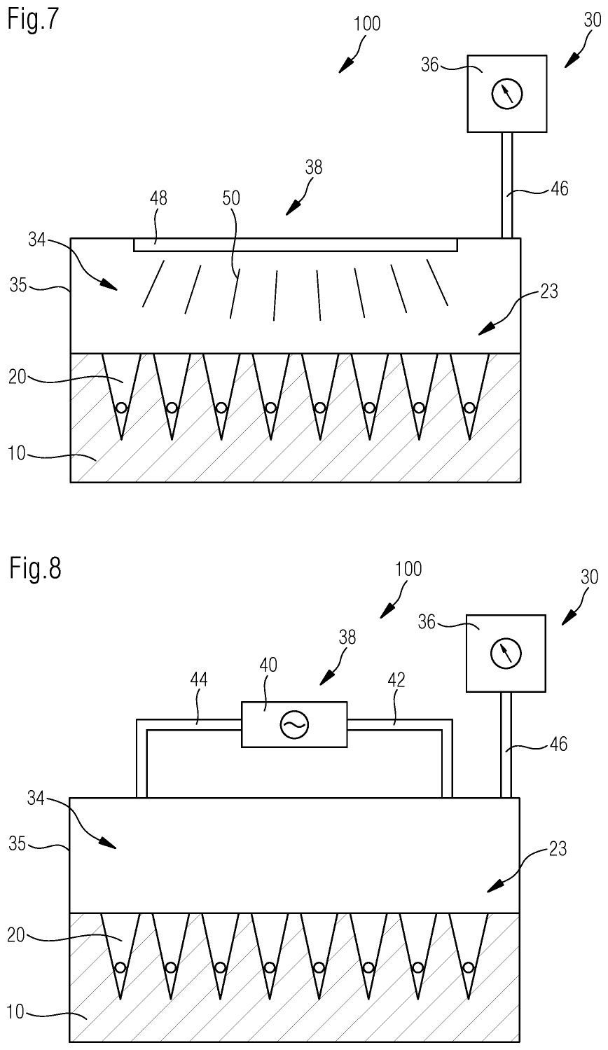

FIGS. 7-9 are schematically sectioned side views of embodiments of devices for manufacturing a microneedle element according to the invention, comprising a die in a condition for illustrating further embodiments of a method for manufacturing microneedle elements according to the invention.

### DESCRIPTION OF THE INVENTION

In the Figures, similar or identical components or elements are identified by the same reference numerals or variations thereof (e.g. 22 and 22′). In particular for improved clarity, preferably already identified elements are not provided with reference numerals in all Figures.

FIGS. 1a-1f and 2 are already described above.

FIG. 3 is based on FIGS. 1a-1f. Here, a state following the state shown in FIG. 1f is shown, but (in FIG. 1f) the backing formulation 20 has not yet hardened and/or dried.

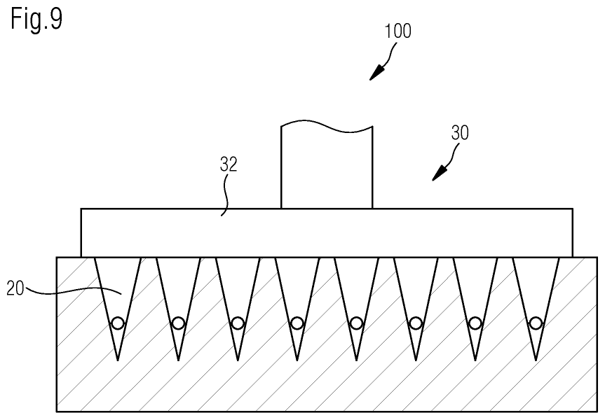

Pressure is applied to the not yet hardened, in particular moist, backing formulation 20 (FIG. 3). This pressurization can be realized by means of physical pressure, e.g. by means of a stamp (cf. FIG. 8) or by means of a roller. A pressurization by means of overpressure, e.g. by means of an overpressure chamber (cf. FIG. 7), is preferred. Said overpressure is in particular approx. 3 bar compared to ambient pressure. The pressurization occurs simultaneously with the curing of the backing formulation 20. Preferably, the pressurization takes place for at least 30 minutes. Thus, a pressure is applied to the backing formulation 20 over a period of time.

The applied pressure causes compression of the air pocket, which in FIG. 3 is an air bubble. The air bubble forms a cavity 24 between backing formulation 20 and tip formulation 14. When baking formulation 20 hardens, in particular dries, the air bubble can no longer expand. A detailed view of this is shown in FIG. 4.

Due to the compression, the air bubble and thus cavity 24 is much smaller than cavities 24′ of previous manufacturing methods (cf. e.g. FIG. 2). This results in a greater bonding contact between backing formulation 20 and tip formulation 14 than in previous manufacturing methods (cf. e.g. FIG. 2). The cross-sectional area of cavity 24 is preferably smaller than the bonding surface between backing formulation 20 and tip formulation 14, in particular by at least 30%. The diameter of the cavity is in particular smaller than the diameter of microneedle 22 at the junction between backing formulation 20 and tip formulation 14, in particular by at least 30%. This minimized cavity 24 or the enlarged bonding contact between backing formulation 20 and tip formulation 14 advantageously leads to an increased stability of microneedle 22 compared to previous microneedles 22′ (cf. e.g. FIG. 2).

The air pressure inside cavity 24 in particular corresponds to the applied overpressure. Preferably, the air pressure in cavity 24 is higher than the ambient pressure. The air pressure in cavity 24 is in particular higher than the air pressure in cavity 24′ of prior art (cf. e.g. FIG. 2).

The microneedles 22 of FIGS. 3 and 4 correspond to an embodiment of microneedle elements according to the invention, wherein several, in particular all microneedles 22 of FIG. 3 preferably correspond to a microarray according to the invention.

FIG. 5 is also substantially based on FIG. 3. In contrast to the embodiment of FIG. 3, microarray 23 in FIG. 5 comprises microneedles 22 with three formulations 14, 20, 26. Here, the cavities 24, 28 are each miniaturized (compared to prior art).

Said small cavities 24, 28 can be implemented, for example, by separately pressurizing twice with simultaneous curing, or by pressurizing the formulations 20, 26 together and curing them simultaneously.

FIG. 6 is also substantially based on FIG. 3. In contrast to the embodiment of FIG. 3, microarray 23 of FIG. 6 is an integral microarray 23. In particular, this can be implemented by overfilling the mold openings 12 with backing formulation 20 so that backing formulation 20 binds together for several microneedles 22 to be manufactured. This bonding surface serves as a backing layer 21 for microarray 23, for example. A pressurization as defined above may be applied to the common backing formulation 20 of the several microneedles 22 to be manufactured.

FIG. 7 is also substantially based on FIG. 3. In addition to the filled mold element 10, a device 100 for manufacturing a microneedle element 23 is shown in FIG. 6.

Device 100 comprises a pressurization device 30. As illustrated, pressurization device 30 comprises a chamber 34, in particular an overpressure chamber, having a chamber wall 35. Chamber 34 encloses mold element 10 at least partially, in particular completely. An overpressure can be applied to backing formulation 20 via a pressure generator 36, in particular a pump, connected to chamber 34. As illustrated, the pressure is applied via line 46, for example.

Optionally, the device (as illustrated) can comprise a drying device 38. The illustrated drying device 38 comprises an air flow generator 40. Air flow generator 40 can in particular comprise a pump and/or a heating element. As illustrated, the drying device is connected to chamber 34 via supply line 42 and discharge line 44. Drying device 38 can be used, for example, to generate an air flow, in particular a warm air flow, for drying baking formulation 20.

Device 100 of FIG. 8 substantially corresponds to device 100 of FIG. 7, wherein another embodiment of drying device 38 is illustrated. The illustrated drying device 38 is a heating element. Here, the backing formulation is heated. This can be done, for example, by a heating device 48 generating infrared rays 50, which is arranged in chamber 34, for example.

FIG. 7 is also substantially based on FIG. 3. A further embodiment of device 100 for manufacturing a microneedle element 23 is illustrated.

Here, pressurization device 30 is a pressing device. The pressing device comprises a stamp 32 applying a physical pressure on the backing formulation.

1. A method for manufacturing a microneedle element comprising at least one microneedle, the method comprising the following steps:
providing a mold element for the microneedle elements to be manufactured;
filling the mold element with a first formulation, in particular a backing formulation,
curing the first formulation, and
pressurizing the first formulation during curing.

2. The method according to claim 1, wherein the method comprises, before the step of filling the mold element with the first formulation, the following further step:
filling the mold element with a second formulation, in particular a tip formulation.

3. The method according to claim 2, wherein the method comprises, after the step of filling the mold element with the second formulation, the following further step:
curing the second formulation, in particular with simultaneous pressurization.

4. The method according to claim 3, wherein the method comprises, before the step of filling the mold element with the first formulation and after the step of filling the mold element with the second formulation, the following further step:
filling the mold element with at least one additional formulation, in particular an intermediate formulation, and preferably
curing the at least one additional formulation, in particular with simultaneous pressurization.

5. The method according to claim 1, wherein, when filling the mold element with the first formulation, the mold element is overfilled so that the first formulation emerges from the mold element.

6. The method according to claim 1, wherein drying, in particular air drying, takes place during curing.

7. The method according to claim 1, wherein temperature is applied during curing.

8. The method according to claim 1, wherein the pressurization is realized by means of overpressure and/or by means of a pressing device, in particular comprising a punch and/or a roller.

9. The method according to claim 1, wherein the pressurization and/or curing is carried out over a period of at least 30 minutes, preferably at least 60 minutes, particularly preferably at least 90 minutes.

10. The method according to claim 1, wherein the mold element comprises several microneedle negative molds, wherein the several microneedle negative molds in particular form a microarray negative mold.

11. The method according to claim 1, wherein the mold element is filled with the first formulation such that the first formulation covers the several microneedle negative molds.

12. A microneedle element, in particular a microneedle or microarray, obtainable by a method according to claim 1.

13. A device for manufacturing a microneedle element comprising at least one microneedle, in particular according to a method according to claim 1, the device comprising:
a mold element for the microneedle elements to be manufactured,
a pressurization device, in particular comprising an overpressure chamber, and
preferably a drying device, in particular generating heat and/or air flow,
wherein the pressurization device is configured such that a pressure is applied to the mold element.
